(19) United States
(12) Patent Application Publication
(10) Pub. No.: US 2024/0347143 A1
(43) Pub. Date: Oct. 17, 2024

(54) METHOD, ELECTRONIC DEVICE, AND COMPUTER PROGRAM PRODUCT FOR DATA PROCESSING

(71) Applicant: Dell Products L.P., Round Rock, TX (US)

(72) Inventors: Zijia Wang, Weifang (CN); Sanping Li, Beijing (CN); Zhen Jia, Shanghai (CN)

(21) Appl. No.: 18/144,291

(22) Filed: May 8, 2023

(30) Foreign Application Priority Data
Apr. 14, 2023 (CN) 202310403340.8

(51) Int. Cl. G16C 20/70; G16C 20/10; G16C 20/20

(52) U.S. Cl. CPC G16C 20/70; G16C 20/10; G16C 20/20

(57) ABSTRACT

Embodiments of the present disclosure provide a method, an electronic device, and a computer program product for data processing. The method may include acquiring a feature representation of state information of a ligand molecule, where the state information comprises at least position information and directional information of the ligand molecule. The method may further include determining, by using a trained reinforcement learning model, additional state information and a feedback value of the ligand molecule based on the feature representation of the state information and a feature representation of state information of a receptor molecule corresponding to the ligand molecule. In addition, the method may further include outputting the additional state information responsive to determining that the feedback value reaches a predetermined threshold. Compared with conventional computer chemistry solutions, the present disclosure can save substantial computational resources and time costs for experimentation, thereby optimizing user experience.

### RELATED APPLICATION

The present application claims priority to Chinese Patent Application No. 202310403340.8, filed Apr. 14, 2023, and entitled “Method, Electronic Device, and Computer Program Product for Data Processing,” which is incorporated by reference herein in its entirety.

### FIELD

Embodiments of the present disclosure relate to the field of computers, and more particularly, to a method, an electronic device, and a computer program product for data processing.

### BACKGROUND

Molecular docking technology is an important process in drug studies and drug design and is used to screen small molecules or ligand molecules for binding to a target protein receptor, so as to alter original biochemical properties of the protein receptor to form a new stable complex. During docking of a ligand molecule to a protein molecule, states (e.g., position, direction, and twist angle) of the ligand molecule are altered to find an ideal binding site on the protein receptor molecule. Because the conformations of the ligand molecule and the receptor molecule are complex, molecular docking is usually implemented by using computing power of computers instead of manpower. However, the computing power and time cost of computers in accomplishing molecular docking tasks are still considerable.

### SUMMARY

Embodiments of the present disclosure provide a data processing solution.

In a first aspect of the present disclosure, a data processing method is provided. The method may include acquiring a feature representation of state information of a ligand molecule, where the state information comprises at least position information and directional information of the ligand molecule. The method may further include determining, by using a trained reinforcement learning model, additional state information and a feedback value of the ligand molecule based on the feature representation of the state information and a feature representation of state information of a receptor molecule corresponding to the ligand molecule. In addition, the method may further include outputting the additional state information responsive to determining that the feedback value reaches a predetermined threshold.

In a second aspect of the present disclosure, an electronic device is provided, including: a processor; and a memory, coupled to the processor and having instructions stored therein, where the instructions, when executed by the processor, cause the electronic device to perform actions including: acquiring a feature representation of state information of a ligand molecule, wherein the state information comprises at least position information and directional information of the ligand molecule; determining, by using a trained reinforcement learning model, additional state information and a feedback value of the ligand molecule based on the feature representation of the state information and a feature representation of state information of a receptor molecule corresponding to the ligand molecule; and outputting the additional state information responsive to determining that the feedback value reaches a predetermined threshold.

In a third aspect of the present disclosure, a computer program product is provided. The computer program product is tangibly stored on a non-transitory computer-readable medium and includes machine-executable instructions. The machine-executable instructions, when executed by a machine, cause the machine to perform any steps of the method according to the first aspect.

This Summary is provided to introduce the selection of concepts in a simplified form, which will be further described in the Detailed Description below. The Summary is neither intended to identify key features or main features of the present disclosure, nor intended to limit the scope of the present disclosure.

### BRIEF DESCRIPTION OF THE DRAWINGS

By describing example embodiments of the present disclosure in more detail with reference to the accompanying drawings, the above and other objectives, features, and advantages of the present disclosure will become more apparent, and identical or similar reference numbers generally represent identical or similar components in the example embodiments of the present disclosure.

In the accompanying drawings:

FIG. 1 shows a schematic diagram of an example environment in which multiple embodiments of the present disclosure can be implemented;

FIG. 2 shows a flow chart of a process for data processing according to embodiments of the present disclosure;

FIG. 3 shows a schematic diagram of a detailed structure of a pretrained feature extraction model according to embodiments of the present disclosure;

FIG. 4 shows a schematic diagram of state information of a ligand molecule according to embodiments of the present disclosure;

FIG. 5 shows a schematic diagram of a detailed architecture for determining additional state information according to embodiments of the present disclosure;

FIG. 6 shows a schematic diagram of a detailed example environment for training an encoder and a decoder according to embodiments of the present disclosure; and

FIG. 7 shows a block diagram of a computing device that can implement multiple embodiments of the present disclosure.

### DETAILED DESCRIPTION

Principles of the present disclosure will be described below with reference to several example embodiments illustrated in the accompanying drawings.

The term “include” and variants thereof used herein indicate open-ended inclusion, that is, “including but not limited to.” Unless specifically stated, the term “or” means “and/or.” The term “based on” means “based at least in part on.” The terms “an example embodiment” and “an embodiment” indicate “a set of example embodiments.” The term “another embodiment” indicates “a group of other embodiments.” The terms “first,” “second,” and the like may refer to different or identical objects. Other explicit and implicit definitions may also be included below.

As mentioned above, molecular docking is a key computational technology in structural bioinformatics and structure-based molecular design. This technology is used to predict the preferred conformation and binding strength of a ligand molecule (usually an organic small molecule) when binding to a protein receptor molecule. This technology not only can be used to predict whether a ligand molecule is tightly bound to a target receptor molecule, but also can be used to understand how it binds, which helps improve the effectiveness and selectivity of binding. The molecular docking technology has a wide range of applications, including drug lead compound optimization, structure-based virtual screening, multi pharmacology prediction, drug repositioning, human variation prediction, protein function prediction, and target druggability evaluation. In terms of operation, the molecular docking technology has two stages: predicting the position, direction, and conformation of the ligand molecule when docked to a target binding site, and predicting an assumed docking state of the ligand molecule and the binding strength to the receptor molecule. One of the most important limitations to the molecular docking technology is low accuracy of conventional prediction of the binding strength.

For the field of molecular docking, in conventional computer chemistry solutions, neural network models such as Alpha Fold can be used to determine the position, direction, twist angle, and other state information of the ligand molecule. However, the reasoning time of neural network models depends on factors such as the length of an amino acid sequence. Therefore, the reasoning process of neural network models will consume a large amount of computational resources and time. This can present significant problems for medical research and drug development in related fields.

In view of this, embodiments of the present disclosure provide a data processing solution. In illustrative embodiments of the solution, a novel model framework is provided that combines a neural network model with a reinforcement learning model. Due to the directionality of the reinforcement learning model, embodiments of the present disclosure can save substantial computational resources and time costs for experimentation compared with conventional computer chemistry solutions, thereby optimizing user experience.

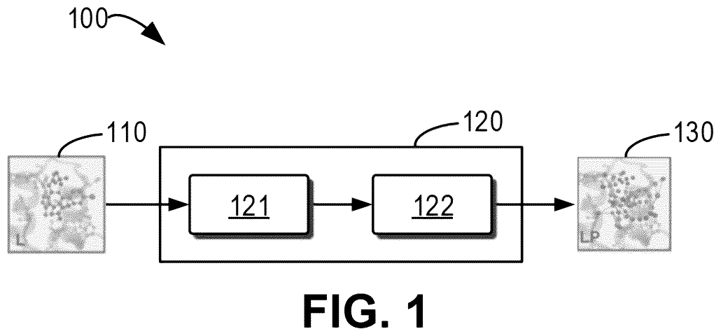

Illustrative embodiments of the present disclosure will be specifically described below with reference to the accompanying drawings. FIG. 1 shows a schematic diagram of an example environment 100 in which multiple embodiments of the present disclosure can be implemented. As shown in FIG. 1, the example environment 100 includes state information 110, a computing device 120, and additional state information 130. In some embodiments, the state information 110 may be position information, directional information, and twist angle information of a ligand molecule for executing molecular docking relative to a receptor molecule. The computing device 120 processes the state information 110 to determine the additional state information 130. The additional state information may be position information, directional information, and twist angle information of a ligand molecule that has basically implemented molecular docking relative to a receptor molecule.

As shown in FIG. 1, the computing device 120 may include a feature extraction model 121 and a reinforcement learning model 122. In some embodiments, the state information 110 is input to the feature extraction model 121, so as to acquire a feature representation (in some embodiments illustratively referred to as an “embedding”) of the state information 110 of the ligand molecule, and the reinforcement learning model 122 may use the feature representation of the state information 110 to determine the additional state information 130 of the ligand molecule that has basically implemented molecular docking.

In some embodiments, the computing device 120 may include, but is not limited to, a personal computer, a server computer, a handheld or laptop device, a mobile device (such as a mobile phone, a personal digital assistant PDA, and a media player), a consumer electronic product, a minicomputer, a mainframe computer, a cloud computing resource, and so on. It should be understood that, based on factors such as cost, the computing device 120 may or may not have sufficient computing resources for model training.

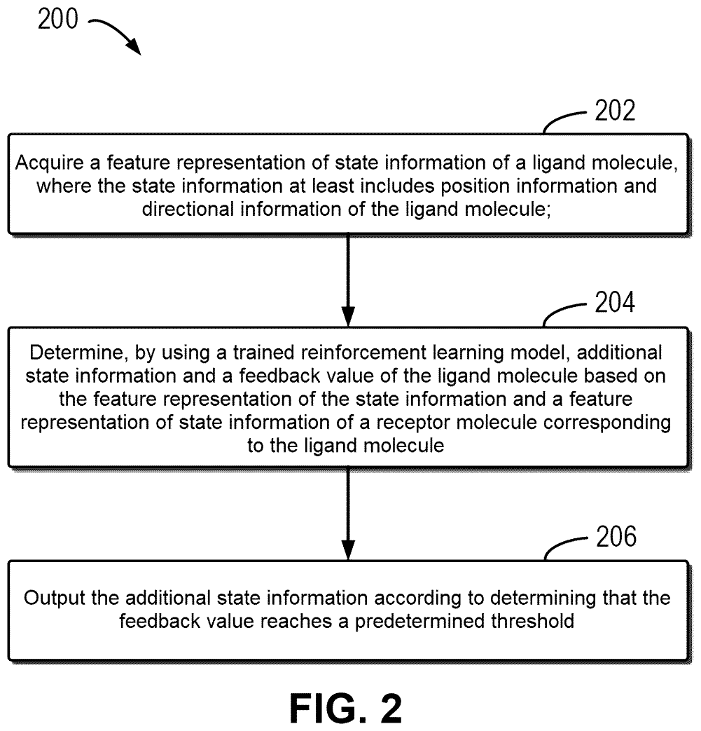

It should be understood that the architecture and functions of the example environment 100 are described for illustrative purposes only, without implying any limitation to the scope of the present disclosure. Embodiments of the present disclosure may also be applied to other environments having different structures and/or functions. In order to explain the principle of the above solution more clearly, the data processing process of the present disclosure will be described in more detail below with reference to FIG. 2.

FIG. 2 shows a flow chart of a process 200 for data processing according to embodiments of the present disclosure. In some embodiments, the process 200 may be implemented in the computing device 120 in FIG. 1 or other computing devices. The process 200 for data processing according to embodiments of the present disclosure is described with reference to FIG. 2 in combination with FIG. 1. For ease of understanding, specific examples mentioned in the following description are all illustrative and are not intended to limit the protection scope of the present disclosure.

At 202, the computing device 120 may acquire a feature representation of the state information 110 of the ligand molecule. It should be understood that the state information may at least include position information and directional information of the ligand molecule. It should be further understood that the computing device 120 may often implement molecular docking by fixing the receptor molecule and adjusting the ligand molecule. Therefore, the computing device 120 herein has generally acquired relevant state information of the receptor molecule, and the processing on relevant data herein may be only for the ligand molecule.

In some embodiments, to acquire the feature representation of the state information 110, the computing device 120 may input the state information 110 of the ligand molecule to the feature extraction model 121. As an example, the feature extraction model 121 may be an Alpha Fold model known in this field or other models. It should be understood that when the Alpha Fold model, for example, is used in the present disclosure, the model is not directly used to generate a final result; instead, an intermediate product of the model is used, that is, the model is used to extract the feature representation of the state information 110.

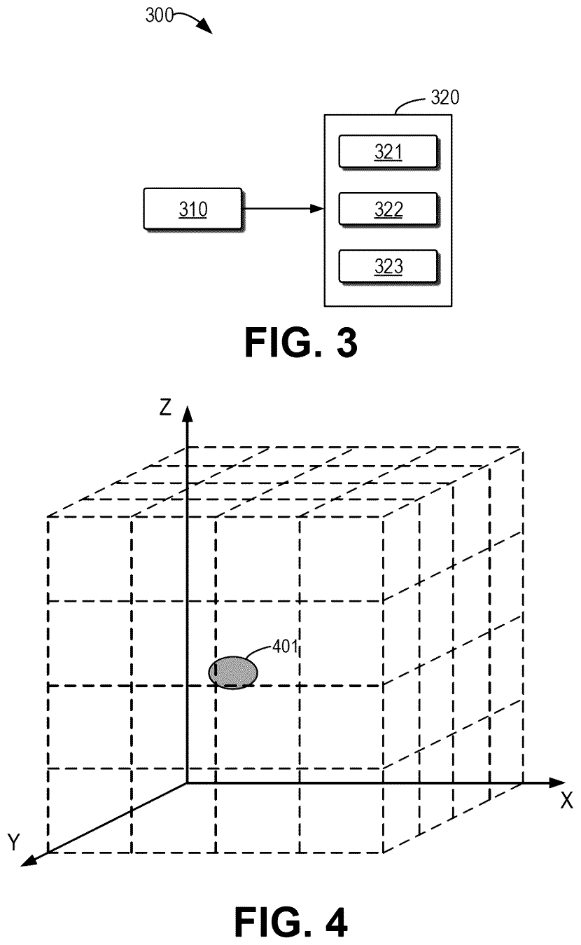

In some embodiments, to improve availability of the feature representation of the state information 110 extracted by, for example, the Alpha Fold model, a training data set and one or more self-supervised models known in this field can be used to pretrain the Alpha Fold model. The training data set mentioned herein can be a data set such as a Protein Data Bank (PDB). In this way, the feature representation output from the feature extraction model 121 is suitable for the subsequent reinforcement learning model 122. To show operations of pretraining on the feature extraction model 121 more clearly, a process for pretraining the Alpha Fold model by using three known self-supervised models is described below with reference to FIG. 3.

FIG. 3 shows a schematic diagram of a detailed architecture 300 of a pretrained feature extraction model according to embodiments of the present disclosure. As shown in FIG. 3, the architecture 300 includes an Alpha Fold model 310 and a self-supervised training module 320. As an example, the self-supervised training module 320 may include one or more self-supervised models. In FIG. 3, the self-supervised training module 320 illustratively comprises a self-predictive representation model 321, a goal-conditioned reinforcement learning model 322, and an inverse dynamics model 323.

The self-predictive representation model 321 is a representation learning algorithm developed for efficient reinforcement learning of data, and may be configured, for example, in accordance with techniques described in Schwarzer et al., “Data-Efficient Reinforcement Learning with Self-Predictive Representations,” ICLR 2021, and Schwarzer et al., “Pretraining Representations for Data-Efficient Reinforcement Learning,” 35^{th} Conference on Neural Information Processing Systems (NeurIPS 2021), which are incorporated by reference herein in their respective entireties.

The self-predictive representation model 321 learns a potential spatial transformation pattern and directly predicts the representation of future states without reconstruction or negative samples. In other words, the self-predictive representation model 321 is used to solve a problem of what is the state of a next state sₒ if an action a is executed in a state s. Specifically, a result predicted by the self-predictive representation model 321 can be compared with a result obtained by calculation according to a predetermined policy so as to determine a loss, such as, for example, a cosine similarity loss. As an example, the self-predictive representation model 321 can learn a convolutional encoder fₒ, so the state can be represented as zₜ=fₒ(sₜ). Then, the self-predictive representation model 321 uses a dynamics model h to evaluate a next state [symbol] =h( [symbol] ,aₜ₊ₖ), starting from [symbol] [symbol] zₜ. These representations are projected to a low dimensional space by means of a projection function pₒ, so as to generate [symbol] [symbol] pₒ( [symbol] ). Meanwhile, the self-predictive representation model 321 uses a target encoder fₘ to generate a target representation [symbol] [symbol] fₘ(sₜ₊ₖ), which is further projected by means of an objective projection function pₘ to generate [symbol] [symbol] pₘ( [symbol] ). Then, the self-predictive representation model 321 uses a learned linear prediction function q to convert ŷ into y̅, so as to maximize the cosine similarity between these predictions and the target, as shown in the following Equation (1):

$${ℒ}_{θ}^{SPR} ( {s}_{t : t + K} , {a}_{t : t + K} ) = - {\sum }_{k = 1}^{K} \frac{q ( {\hat{y}}_{t + k} ) \cdot {\overline{y}}_{t + k}}{{‖ q ( {\hat{y}}_{t + k} ) ‖}_{2} \cdot {‖ {\overline{y}}_{t + k} ‖}_{2}} \qquad ( 1 )$$

Parameters of these target models θₘ are defined as an exponential moving average of parameters θₒ of fₒ and pₒ: θₘ=τθₘ+(1−τ)θₒ.

The goal-conditioned reinforcement learning model 322 indicates that modeling many different value functions is a useful representation learning target. See, for example, Dabney et al., “The Value-Improvement Path: Towards Better Representations for Reinforcement Learning,” 2021, which is incorporated by reference herein in its entirety. Therefore, the self-predictive representation model 321 can be reinforced. In other words, the goal-conditioned reinforcement learning model 322 is used to solve a problem of what is an action a that should be executed if a state s is converted to a state s₀. Specifically, a result predicted by the goal-conditioned reinforcement learning model 322 can be compared with a result obtained by calculation according to a predetermined policy so as to determine a loss. As an example, the loss can be a cosine similarity loss.

The inverse dynamics model 323 is used to solve a problem of what action a can be adopted to achieve the fastest conversion from a state s to a state s₀. See, for example, Lesort et al., “State Representation Learning for Control: An Overview,” Neural Networks, 2018, which is incorporated by reference herein in its entirety.

Due to the fact that features extracted by neural networks such as an Alpha Fold model are usually not suitable for reinforcement learning, the above self-supervised model can be used to pretrain the Alpha Fold model, thereby improving the overall prediction accuracy of the model.

When pretraining of the feature extraction model 121 is finished, the training data set can be used to further train the reinforcement learning model 122. Similar to conventional reinforcement learning training, a training environment can be created. Relevant data of the ligand molecule in the training data set is collected from the training environment, the data is preprocessed, and the preprocessed data is finally used to train the reinforcement learning model 122. As an example, relevant data of the ligand molecule in the training data set can be input to the pretrained feature extraction model 121 to extract the feature representation, so as to further input the feature representation to the reinforcement learning model 122, thereby implementing the training of the reinforcement learning model 122.

Returning to FIG. 2, at 204, the computing device 120 may determine, by using a trained reinforcement learning model, additional state information and a feedback value of the ligand molecule based on the feature representation of the state information and a feature representation of state information of a receptor molecule corresponding to the ligand molecule.

In some embodiments, the reinforcement learning model is a Q learning model; moreover, during a process of training the model, the computing device 120 may input state information and action information in the training data set to the Q learning model so as to determine a corresponding Q value and update the Q learning model based on the Q value. It should be understood that the Q value can be determined at least based on a learning rate, a feedback function value, and a Q value of best action information for converting to next state information.

Q learning is a reinforcement learning algorithm that maps every attempt of an agent to each state to a Q value or Q(s, a). Q(s, a) indicates a value of executing an action a at a specific state s. Since the agent does not know any information before exploring a surrounding environment, it performs random actions from its initial state. If the agent performs enough attempts during the interaction with the environment, it can learn a real value of Q (s, a) and update Q (s, a) using the following Equation (2):

$$Q ( s , a ) ← Q ( s , a ) + α [ r + γ {max}_{a} \cdot Q ( {s}^{′} , {a}^{′} ) - Q ( s , a ) ] \qquad ( 2 )$$

where α represents a learning rate, r represents a received feedback value (that is, a reward), γ represents a discount rate, and Q(s′, a′) represents a value of the best action of a next state. Here, the range of the learning rate α is between 0 and 1. If α is close to 1, the agent will consider more of the gained learning experience and at least partially override the existing action values. It should be understood that an environment for reinforcement learning is illustratively a three-dimensional position, direction, and twist angle of protein receptor molecules and ligand molecules. The agent can be considered as a robot exploring these variable spaces and learning to adjust values to find an optimal value. By arranging the reinforcement learning model 122, the process of molecular docking will be directional rather than random attempts, so that the computational amount and time can be significantly reduced.

In some embodiments, the action information may be an operation adopted by the ligand molecule to convert from current state information to next state information, and the operation can be: moving a root atom of the ligand molecule by a predetermined distance, rotating the ligand molecule by a predetermined angle, and/or twisting the ligand molecule by a predetermined twist angle. It should be understood that state information of the agent used to imitate the ligand molecule can be a root position, direction, and twist angle of the ligand molecule in a three-dimensional space. These variables represent the state of the ligand molecule at any specific time during the optimization process. As an example, position variables (x, y, z) can be discretized by dividing the space into isometric grids or cubes with a granularity of 0.2. FIG. 4 shows a schematic diagram of state information of a ligand molecule according to embodiments of the present disclosure.

As shown in FIG. 4, a root atom 401 of a ligand is located in the center of a box representing a search space. The search space is discretized in each axis direction. As an example, as shown in FIG. 4, the search space is divided into 4 parts on the x axis, 4 parts on the y axis, and 4 parts on the z axis. Therefore, there may be 64 discrete positions in the search space. In this way, the numerical representation of the position information in the state information can be achieved. Similarly, rotation vectors [r₁, r₂, r₃]^{T} of the direction angle of the ligand molecule are discretized into values of equal size, and the step size of the rotation angle can be selected as 5°. The twist angle can also be represented in a similar way.

Therefore, a set S of all possible state information is a combination of all discretized position information, directional information, and twist angle information: S=P×O×T, where P is a set of discretized root positions of the ligand molecule, O is a set of discretized directions of the ligand molecule, and T is a set of discretized twist angles of the ligand molecule.

In addition, the action information of the agent can be represented in the following manner. As an example, the position of the root atom can be moved. The agent may convert an initial position of the root atom to another position in any octant within 0.2. Therefore, the new position can be determined by the following set of spherical coordinate equations collectively referred to below as Equation (3):

$$x = {x}_{0} + r * sin ( θ ) cos ( φ ) \qquad ( 3 )$$

$$y = {y}_{0} + r * sin ( θ ) sin ( φ )$$

$$z = {z}_{0} + r * cos ( φ )$$

where θ is an angle between the positive x axis and a motion line, p is an angle between the positive z axis and the motion line, and r is a distance increment of 0.2. As another example, an azimuth of the ligand molecule can also be increased or decreased. As a further example, the twist angle of the ligand molecule can also be increased or decreased.

In addition, a reward function (that is, a feedback value function) involved in the Q learning algorithm or the reinforcement learning algorithm can be determined based on a conventional Vina scoring function. When the conformation gives a low energy value δ, a higher reward value is required, as shown in the following Table 1.

TABLE 1

| Reward value | Condition |
|---|---|
| 1 | δ > −0.2 |
| 5 | −0.5 < δ ≤ −0.2 |
| 100 | δ < −0.5 |
| −1 | δ < 0.2 |
| −5 | 0.2 ≤ δ < 0.5 |

Returning again to FIG. 2, at 206, the computing device 120 may output the additional state information 130 when determining that the feedback value reaches a predetermined threshold. In this way, quick and exact molecular docking can be implemented.

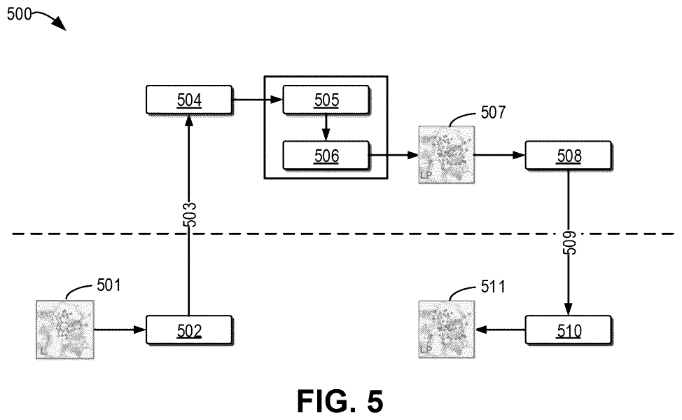

FIG. 5 shows a schematic diagram of a detailed architecture 500 for determining additional state information according to embodiments of the present disclosure. It should be understood that to reduce computing power requirements of an edge computing node, most of the reasoning work can usually be transferred to a server side (or a cloud end). As shown in FIG. 5, an area above the dashed line represents the server side, while an area below the dashed line represents the edge computing node side. Therefore, when state information 501 of the ligand molecule is received at the edge computing node, an encoder 502 arranged at the edge computing node can be used to determine a compressed version 503 of the state information 501 of the ligand molecule. It should be understood that the encoder 502 can be a trained convolutional neural network model. In this way, the size of the state information of the ligand molecule can be reduced for subsequent transmission.

Then, the compressed version 503 will be sent to the server side, so that a decoder 504 arranged at the server side is caused to determine a decompressed version of the compressed version 503. Then, similar to the previous data processing process, a decompressed version of the state information 501 is input to a feature extraction model (for example, Alpha Fold) 505 arranged at the server side for executing feature extraction, and the extracted feature representation is further input to a trained reinforcement learning (for example, Q learning) model 506, so as to determine state information 507 of a next state that basically implements molecular docking. It should be understood that the feature extraction model 505 and the reinforcement learning model 506 can be separately set or encapsulated integrally.

Then, at the server side, an encoder 508 is used to determine a compressed version 509 of the state information 507, and the compressed version 509 is transmitted back to the edge computing node, so as to determine a decompressed version 511 of the compressed version 509 by using a decoder 510 arranged at the edge computing node. It should be understood that the decompressed version 511 generated at the edge computing node is an optimal or better state of the ligand molecule that is suitable for performing molecular docking. Based on the state information, the ligand molecule can be docked to a target receptor molecule.

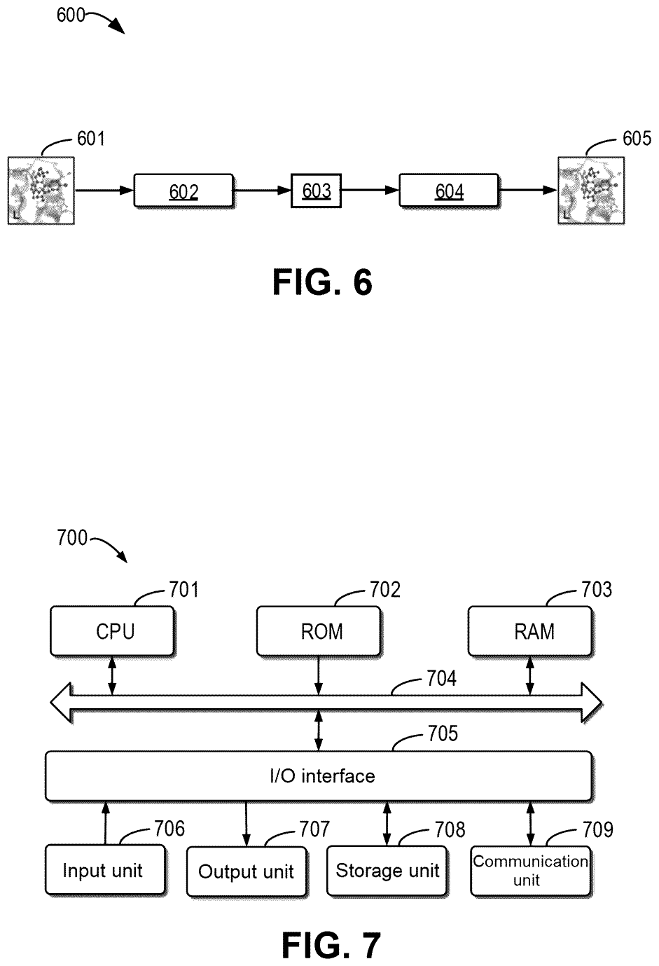

It should be understood that the encoders 502 and 508 in FIG. 5 can have the same parameters, and the decoders 504 and 510 can have the same parameters. To show processes of training these encoders and decoders, FIG. 6 further shows a schematic diagram of a detailed example environment 600 for training an encoder and a decoder according to embodiments of the present disclosure.

As shown in FIG. 6, state information 601 acquired from a training data set is input to an encoder 602 so as to determine a compressed version 603, and the compressed version 603 is further input to a decoder 604 so as to determine a decompressed version 605. At this time, parameters of the encoder 602 and the decoder 604 can be adjusted based on a difference between the compressed version 603 and the decompressed version 605 until convergence is achieved.

By means of the above embodiments, the present disclosure provides a new architecture based on a feature extraction model and a reinforcement learning model, so as to implement molecular docking more efficiently. In addition, using multiple self-supervised models to pretrain the feature extraction model can make features extracted by the feature extraction model more suitable for reinforcement learning. In addition, in the present disclosure, main computing tasks such as reasoning are placed at a server side or in a cloud, thereby reducing the computing power requirements of an edge computing node.

FIG. 7 illustrates a block diagram of a computing device 700 that can be configured to implement a plurality of embodiments of the present disclosure. The computing device 700 is an example of what is more generally referred to herein as an “electronic device.” Electronic devices as that term is generally used herein are intended to represent various forms of digital computers, such as laptop computers, desktop computers, workbenches, personal digital assistants, servers, blade servers, mainframe computers, and other suitable computers. Electronic devices may also represent various forms of mobile devices, such as personal digital processing devices, cellular telephones, smartphones, wearable devices, and other similar computing devices. Components shown herein, their connections and relationships, and their functions are only used as examples and are not intended to limit the embodiments of the present disclosure described and/or claimed herein.

As shown in FIG. 7, computing device 700 includes a computing unit 701, illustratively implemented at least in part as a central processing unit (CPU), that may perform various appropriate actions and processing according to a computer program stored in a read-only memory (ROM) 702 or a computer program loaded from a storage unit 708 to a random access memory (RAM) 703. Various programs and data required for the operation of the computing device 700 may also be stored in the RAM 703. The computing unit 701, the ROM 702, and the RAM 703 are connected to each other via a bus 704. An Input/Output (I/O) interface 705 is also connected to the bus 704.

A plurality of components in the computing device 700 are connected to the I/O interface 705, including: an input unit 706, such as a keyboard and a mouse; an output unit 707, such as various types of displays and speakers; the storage unit 708, such as a magnetic disk and an optical disc; and a communication unit 709, such as a network card, a modem, and a wireless communication transceiver. The communication unit 709 allows the computing device 700 to exchange information/data with other devices via a computer network, such as the Internet, and/or various telecommunication networks.

The computing unit 701 may be various general-purpose and/or special-purpose processing components with processing and computing capabilities. Some examples of the computing unit 701 include, but are not limited to, CPUs, graphics processing units (GPUs), various specialized artificial intelligence (AI) computing chips, various computing units for running machine learning model algorithms, digital signal processors (DSPs), and any appropriate processors, controllers, microcontrollers, etc. The computing unit 701 performs the various methods and processing described above, such as a process 200. For example, in some embodiments, the process 200 may be implemented as a computer software program that is tangibly included in a machine-readable medium, for example, the storage unit 708. In some embodiments, part or all of the computer program may be loaded and/or installed onto the computing device 700 via the ROM 702 and/or the communication unit 709. When the computer program is loaded to the RAM 703 and executed by the computing unit 701, one or more steps of the process 200 described above may be performed. Alternatively, in other embodiments, the computing unit 701 may also be configured to implement the process 200 in any other suitable manner (such as by means of firmware).

Various embodiments of the systems and techniques described herein above may be implemented in a digital electronic circuit system, an integrated circuit system, a field programmable gate array (FPGA), an application specific integrated circuit (ASIC), an application specific standard product (ASSP), a system on a chip (SOC), a complex programmable logic device (CPLD), computer hardware, firmware, software, and/or combinations thereof. These embodiments include various implementations in which the disclosed processing and methods are performed in one or more computer programs which can be executed and/or interpreted on a programmable system including at least one programmable processor, and the programmable processor can be a special-purpose or general-purpose programmable processor, which can receive data and instructions from a storage system, at least one input device, and at least one output device, and transmit the data and instructions to the storage system, the at least one input device, and the at least one output device.

Program code for implementing methods of the present disclosure may be written by using one programming language or any combination of a plurality of programming languages. The program code may be provided to a processor or controller of a general purpose computer, a special purpose computer, or another programmable data processing apparatus, such that the program code, when executed by the processor or controller, implements the functions/operations specified in the flow charts and/or block diagrams. The program code may be executed completely on a machine, executed partially on a machine, executed partially on a machine and partially on a remote machine as a stand-alone software package, or executed completely on a remote machine or server.

In the context of the present disclosure, a machine-readable medium may be a tangible medium that may include or store a program for use by an instruction execution system, apparatus, or device or in connection with the instruction execution system, apparatus, or device. The machine-readable medium may be a machine-readable signal medium or a machine-readable storage medium. The machine-readable medium may include, but is not limited to, an electronic, magnetic, optical, electromagnetic, infrared, or semiconductor system, apparatus, or device, or any suitable combination thereof. More specific examples of the machine-readable storage medium may include one or more wire-based electrical connections, a portable computer diskette, a hard disk, a RAM, a ROM, an erasable programmable read-only memory (EPROM or flash memory), an optical fiber, a portable compact disk read-only memory (CD-ROM), an optical storage device, a magnetic storage device, or any suitable combinations thereof.

To provide interaction with a user, the systems and techniques described herein can be implemented on a computer having: a display device (for example, a cathode ray tube (CRT) or liquid crystal display (LCD) monitor) for displaying information to a user; and a keyboard and a pointing device (for example, a mouse or a trackball) through which a user can provide input to the computer. Other kinds of devices may also be used to provide interaction with the user; for example, the feedback provided to the user can be any form of sensory feedback (such as visual feedback, auditory feedback, or tactile feedback); and additionally, input from the user may be received in any form (including acoustic input, voice input, or tactile input).

The systems and techniques described herein can be implemented on a computing system that includes back-end components (e.g., as a data server), or a computing system that includes middleware components (e.g., an application server), or a computing system that includes front-end components (e.g., a user computer having a graphical user interface or a web browser through which a user can interact with embodiments of the systems and techniques described herein), or a computing system including any combination of such back-end components, middleware components, or front-end components. The components of the system may be mutually connected through digital data communication (for example, a communication network) through any form or medium. An example of the communication network includes: a local area network (LAN), a wide area network (WAN), and the Internet.

The computer system may include a client terminal and a server. The client terminal and the server are generally remote from each other and usually interact through a communication network. A relationship between the client terminal and the server is generated by computer programs that run on corresponding computers and have a client terminal-server relationship with each other.

It should be understood that steps may be reordered, added, or deleted using the various forms of processes shown above. For example, the steps recorded in the present disclosure may be performed in parallel, may be performed sequentially, or may be performed in different orders as long as the desired results of the technical solution disclosed by the present disclosure are achieved, and there is no restriction herein.

The above-described illustrative embodiments do not constitute a limitation to the protection scope of the present disclosure. It will be apparent to those skilled in the art that various modifications, combinations, sub-combinations, and substitutions may be performed according to design requirements and other factors. Any modifications, equivalent replacements, and improvements made within the spirit and principles of the present disclosure shall be included in the scope of protection of the present disclosure.

What is claimed is:

1. A method for data processing, comprising:
acquiring a feature representation of state information of a ligand molecule, wherein the state information comprises at least position information and directional information of the ligand molecule;
determining, by using a trained reinforcement learning model, additional state information and a feedback value of the ligand molecule based on the feature representation of the state information and a feature representation of state information of a receptor molecule corresponding to the ligand molecule; and
outputting the additional state information responsive to determining that the feedback value reaches a predetermined threshold.

2. The method according to claim 1, wherein acquiring the feature representation comprises:
inputting the state information of the ligand molecule to a feature extraction model to acquire the feature representation.

3. The method according to claim 2, wherein the feature extraction model is trained by using a training data set and one or more self-supervised models, so as to cause the feature representation output from the feature extraction model to be suitable for the reinforcement learning model.

4. The method according to claim 1, wherein the reinforcement learning model is a Q learning model, and the method further comprises:
inputting state information and action information in a training data set to the Q learning model so as to determine a corresponding Q value; and
updating the Q learning model based on the Q value,
wherein the Q value is determined at least based on a learning rate, a feedback function value, and a Q value of best action information for converting to next state information.

5. The method according to claim 4, wherein the state information further comprises twist angle information of the ligand molecule.

6. The method according to claim 5, wherein the action information is an operation adopted by the ligand molecule to convert from current state information to the next state information, and the operation comprises at least one of the following operations:
moving a root atom of the ligand molecule by a predetermined distance;
rotating the ligand molecule by a predetermined angle; and
twisting the ligand molecule by a predetermined twist angle.

7. The method according to claim 1, wherein acquiring the feature representation is executed at a server side, and the method further comprises:
determining, at an edge computing node, a compressed version of the state information of the ligand molecule by using an encoder;
sending the compressed version to the server side, so as to determine a decompressed version of the compressed version by using a decoder; and
acquiring the feature representation from the decompressed version.

8. The method according to claim 7, further comprising:
determining, at the server side, an additional compressed version of the additional state information by using the encoder; and
sending the additional compressed version to the edge computing node, so as to determine a decompressed version of the additional compressed version by using the decoder.

9. The method according to claim 1, further comprising:
docking the ligand molecule to the receptor molecule based on the additional state information.

10. An electronic device, comprising:
a processor; and
a memory coupled to the processor and having instructions stored therein, wherein the instructions, when executed by the processor, cause the electronic device to perform actions comprising:
acquiring a feature representation of state information of a ligand molecule, wherein the state information comprises at least position information and directional information of the ligand molecule;
determining, by using a trained reinforcement learning model, additional state information and a feedback value of the ligand molecule based on the feature representation of the state information and a feature representation of state information of a receptor molecule corresponding to the ligand molecule; and
outputting the additional state information responsive to determining that the feedback value reaches a predetermined threshold.

11. The electronic device according to claim 10, wherein acquiring the feature representation comprises:
inputting the state information of the ligand molecule to a feature extraction model to acquire the feature representation.

12. The electronic device according to claim 11, wherein the feature extraction model is trained by using a training data set and one or more self-supervised models, so as to cause the feature representation output from the feature extraction model to be suitable for the reinforcement learning model.

13. The electronic device according to claim 10, wherein the reinforcement learning model is a Q learning model, and the actions further comprise:
inputting state information and action information in a training data set to the Q learning model so as to determine a corresponding Q value; and
updating the Q learning model based on the Q value,
wherein the Q value is determined at least based on a learning rate, a feedback function value, and a Q value of best action information for converting to next state information.

14. The electronic device according to claim 13, wherein the state information further comprises twist angle information of the ligand molecule.

15. The electronic device according to claim 14, wherein the action information is an operation adopted by the ligand molecule to convert from current state information to the next state information, and the operation comprises at least one of the following operations:
moving a root atom of the ligand molecule by a predetermined distance;
rotating the ligand molecule by a predetermined angle; and
twisting the ligand molecule by a predetermined twist angle.

16. The electronic device according to claim 10, wherein acquiring the feature representation is executed at a server side, and the actions further comprise:
determining, at an edge computing node, a compressed version of the state information of the ligand molecule by using an encoder;
sending the compressed version to the server side, so as to determine a decompressed version of the compressed version by using a decoder; and
acquiring the feature representation from the decompressed version.

17. The electronic device according to claim 16, further comprising:
determining, at the server side, an additional compressed version of the additional state information by using the encoder; and
sending the additional compressed version to the edge computing node, so as to determine a decompressed version of the additional compressed version by using the decoder.

18. The electronic device according to claim 10, further comprising:
docking the ligand molecule to the receptor molecule based on the additional state information.

19. A computer program product that is tangibly stored on a non-transitory computer-readable medium and comprises machine-executable instructions, wherein the machine-executable instructions, when executed by a machine, cause the machine to perform actions comprising:
acquiring a feature representation of state information of a ligand molecule, wherein the state information comprises at least position information and directional information of the ligand molecule;
determining, by using a trained reinforcement learning model, additional state information and a feedback value of the ligand molecule based on the feature representation of the state information and a feature representation of state information of a receptor molecule corresponding to the ligand molecule; and
outputting the additional state information responsive to determining that the feedback value reaches a predetermined threshold.

20. The computer program product according to claim 19, wherein acquiring the feature representation comprises:
inputting the state information of the ligand molecule to a feature extraction model to acquire the feature representation.
